(19) United States
(12) Patent Application Publication
(10) Pub. No.: US 2002/0156385 A1
(43) Pub. Date: Oct. 24, 2002

(54) Differentiation of CAD vs NCI with different patterns of empi indexes

(76) Inventors: Genquan Feng, New York, NY (US); Joseph T. Shen, Sands Point, NY (US); Li Feng, Elmhurst, NY (US)

Correspondence Address: Prof. Genquan Feng, P.O. Box 1796, New York, NY, 10185-0016, US

(21) Appl. No.: 09/841,559

(22) Filed: Apr. 24, 2001

(51) Int. Cl. A61B 5/04

(52) U.S. Cl. 600/515

(57) ABSTRACT

Non-invasive to early detect myocardial inschemia is a very important problem in the medical profession. The invention used the method introduced in U.S. Pat. No. 5,509,425 and U.S. Pat. No. 5,649,544 to acquire positive (occurrence) indexes in every case of a sufficient amount database. Used the database empirically screening the said EMPI indexes, to select the CAD related indexes “Ic” and the NCI related indexes “In”, according to the positive rate of those indexes in the CAD patients and NCI patients which one is higher. Combined the “Ic” and “In” as “one Group” (Cluster), namely “Icn” to get the “index patterns” constructed by “Icn”, called “Pcn”. Then used the batabase empirically differentiating the “Pcn” to two Groups, one is “CAD related Group”, called “Pc”, and another is “NCI related Group”, called “Pn”, according to the positive rate of those “index patterns” in the CAD patients and NCI patients which one is higher. Then grossly identified the patient with the index pattern(s) within the scope of the Group “Pc” as a CAD patient, and grossly identified the patient as NCI patient when he has the index pattern(s) within the scope of the Group “Pn”. Finally, optimize the “gross diagnosis”, according to the principle of the method introduced in U.S. Pat. No. 5,542,429, to get final results (final differential diagnosis suggestions).

### BACKGROUND OF INVENTION

1. Field of the Invention

This invention generally relates to a method of, and an arrangement (means) for differentiation of CAD (Coronary Arterial Disease) vs NCI (Non-CAD Ischemia) in human with different patterns of “EMPI indexes” (EKG Multiphase Information Diagnosis Indexes) (see U.S. Pat. No. 5,509,425 issued Apr. 23, 1996 and U.S. Pat. No. 5,649,544 issued Jul. 22, 1997), (thereafter called “EMPI indexes”).

[Note: In this patent, the term “EMPI indexes” defined as: “a selected suitable version (mode) of the indexes of EMPI acquired and defined according to the principle of the method and arrangement of U.S. Pat. No. 5,509,425 and U.S. Pat. No. 5,649,544”. The term “suitable version” defined as: “a version suited to be used in this patent, including but not limited to, new version, advanced version, several modes, models”].

2. Description of the Related Art

Cardiovascular dysfunction, especially myocardial ischemia, is still the leading cause of death in the world and in the United States today. It affects 13.8 million (6.75 million women and 6.93 million men) and debilitates nearly 4.5 million Americans and causes approximately 900,000 deaths, i.e. 48% of the deaths, in the USA and 2.5 million in the world annually. It was estimated that nearly 58 million people suffered one kind of cardiovascular dysfunction, with especially high risk for males. The main cause of cardiac death is acute myocardial ischemia. However, the accuracy of diagnosis of myocardial ischemia is about 50% for the resting EKG. The diagnosis accuracy of many improved methods such as stress test, Holter monitor, late potentials, EKG mapping, echocardiogram. Thallium scan, PET, MRI. CT, etc. for myocardial ischemia are around 65% to 75%. Angiogram and IVUS can reach an accuracy around 80% for detection of CAD, but both are invasive and expensive. A widely quoted acceptable mortality rate for angiography is 0.1%, while the damage rate shall be 10 times or more. Therefore, non-invasively early detecting myocardial ischemia is a very urgent problem in the medical profession curcently. U.S. Pat. No. 5,509,425 and U.S. Pat. No. 5,649,544 have taught us a new technology, EMPI, for early detection of myocardial ischemia in resting, its accuracy is around 93%.

However, myocardial ischemia has two different etiological causes, one type is on the cause of CAD, others are on the cause of non-CAD causes (NCI). For clinical purpose, differentiation of the two different etiological groups is very important for medical treatment and management of the patients. This invention represents a new progress of the EMPI Technology, to use “EMPI indexes” patterns to differentiate CAD patients (including “pure CAD” patients and “CAD combined NCI” patients) vs NCI (“pure NCI no CAD”) patients

### SUMMARY OF THE INVENTION

1. Objects of the Invention

It is a general object of this invention to advance the state of the diagnostic art for detecting different types of the myocardial ischemia, namely CAD and NCI. based on “EMPI indexes” (U.S. Pat. No. 5,509,425 and U.S. Pat. No. 5,649,544). Still another object of this invention is to non-invasively and accurately to diagnose the two different types of myocardial ischemia with “EMPI index” patterns.

2. Feature of the Invention

In keeping with these objects and others, which will become apparent hereinafter, one feature of this invention relates. In its broadest aspect, to determining a condition of a sample by acquiring electrical analog signals from the sample using the principle of the method and means introduced in the two patents (U.S. Pat. No. 5,509,425 and U.S. Pat. No. 5,649,544). In one preferred embodiment, the signals are EKG signals obtained from a surface of the body of a patient by placement of a plurality of surface electrodes at various sites thereon, as introduced in the two US Patent mentioned above. Then use those “EMPI indexes” patterns, obtained from the method mentioned above, to differentiate different types of the dysfunction found.

### BRIEF DESCRIPTION OF THE DRAWINGS

FIG. 1. Screening and acquiring the CAD related indexes group (cluster) “Ic” and NCI related indexes group (cluster) “In” from the whole group of EMPI indexes “I”.

FIG. 2. Put the group “Ic” and “In” together as one group, called “Icn”, which is the “EMPI indexes group (cluster) used for differentiation of CAD and NCI”.

FIG. 3. Empirically screening patterns constructed by “Icn”, (called “PIcn”), to get the patterns “related to CAD”, called group “PIc”, and the patterns “related to NCI”, called group “PIn”.

FIG. 4. Put the “PIc” and the “PIn” together to get whole group of the patterns used for differentiation of CAD and NCI, called “PDcn”.

FIG. 5. A case with “PIc” pattern expresses “he is a CAD patient”. A case with “PIn” pattern means “he is an NCI patient”. That is the method to grossly differentiate CAD and NCI patients. Then use “optimization process” (U.S. Pat. No. 5,542,429) to get the final results.

[Note: In this patent the term “optimization process” defined as: “a selected or revised version of the optimization process, according to the principle of the method and arrangements of U.S. Pat. No. 5,542,429. The term “selected or revised version” defined as a suitable version or a revised version which suited to be used in this patent, including but not limited to, a set of selected suitable factors, indexes, weights, coefficients, parameters, weights of indexes].

### DETAIL DESCRIPTION OF THE PREFERRED EMBODIMENTS

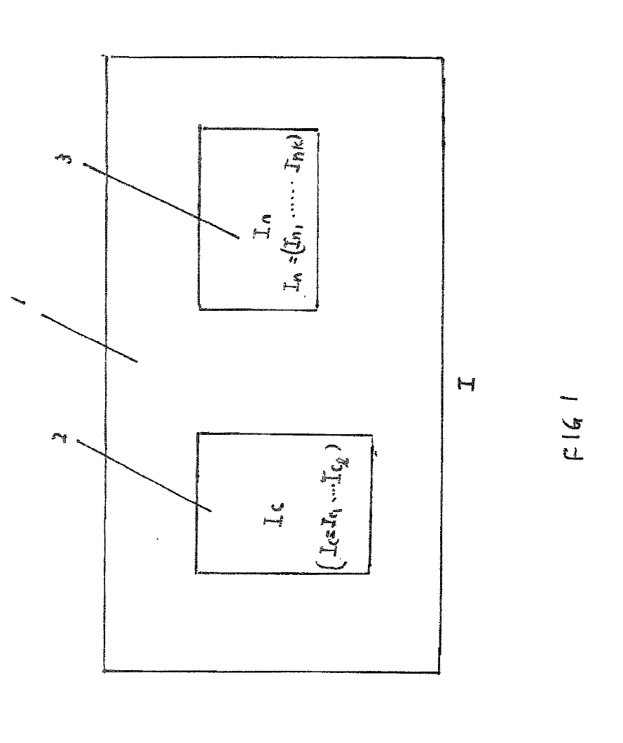

Referring now to the drawings, FIG. 1., Reference numeral 1 generally identifies the whole group of “EMPI indexes” (U.S. Pat. No. 5,649,544 and U.S. Pat. No. 5,509,425). Then empirically “screen” the “EMPI indexes” to acquire two special indexes groups (clusters). One is group “Ic”, reference numeral 2, which is the indexes related to CAD. The other one is the group “In”, reference numeral 3, which is the indexes related to NCI. The definition of (i.e. the screening method of) “Ic” is the indexes of which, its positive (occurrence) rate in CAD patients is higher than NCI patients. The definition of “In” is the indexes of which, its positive (occurrence) rate in NCI patients is higher than CAD patients.

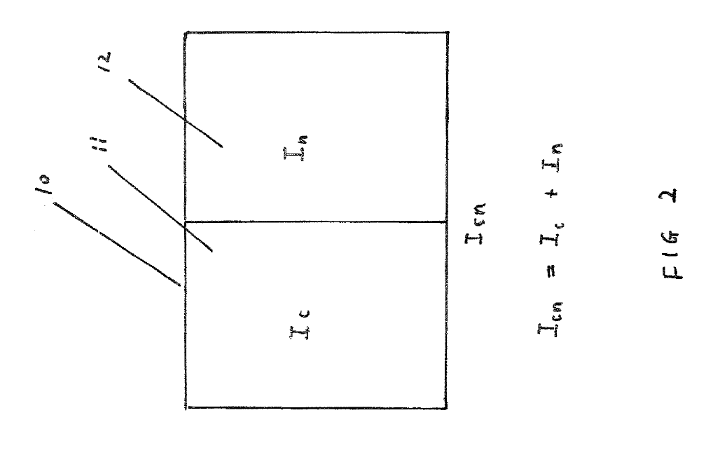

In FIG. 2. Reference numeral 11 generally identifies the group of “EMPI indexes” related to differentiation of CAD vs. NCI, namely “Icn”, which is the combination of the group “Ic” and “In”, reference numeral 11 and 13 respectively.

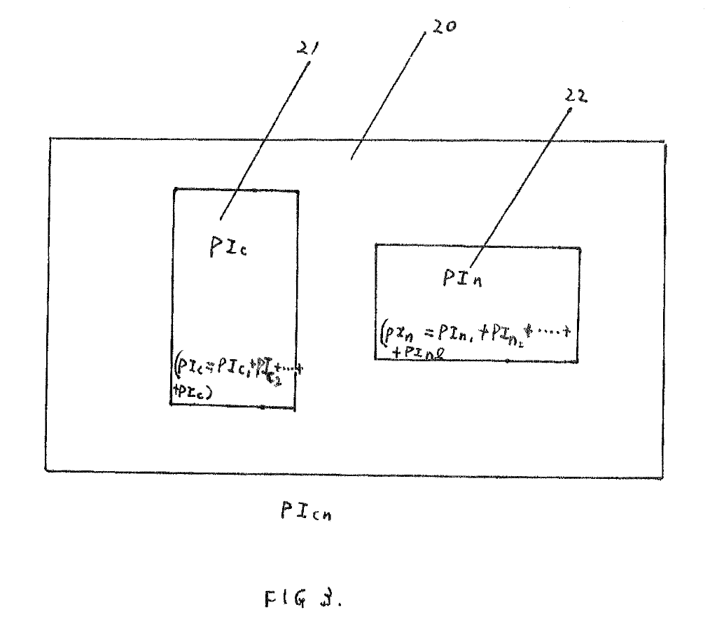

In FIG. 3. Reference numeral 20 generally identifies the group of the index patterns constructed by the “Icn”. Then empirically screen it, to get two groups (clusters) of the index patterns. One is the group “PIc”, reference numeral 21, which is the “index patterns” related to CAD. The other is the group “PIn”, reference numeral 22, which is the “index patterns” related to NCI. The definition (i.e. the screening method) of “PIc” is: the “index patterns” of which, its positive (occurrence) rate in CAD patients is higher than NCI patients. The definition of “PIn” is: the “index patterns” of which, its positive (occurrence) rate in NCI patients is higher than CAD patients.

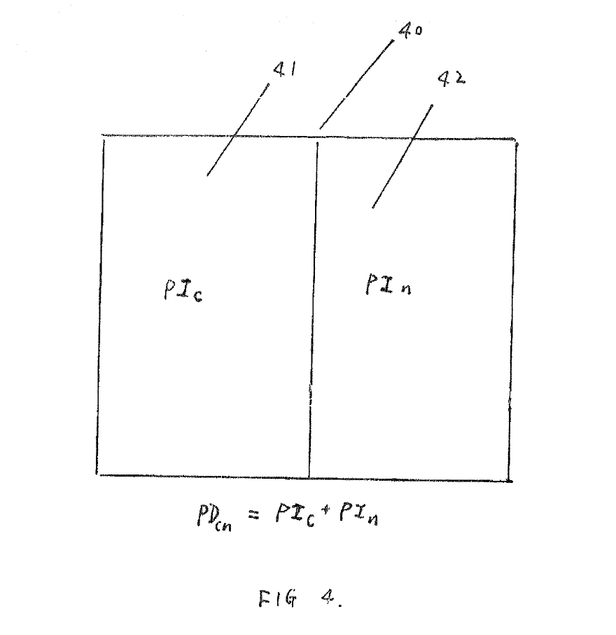

In FIG. 4. Reference numeral 40 identifies the group of the “index patterns” used by the invention for differentiation of CAD and NCI, called “PDcn”. “PDcn is the group of “PIc” (reference numeral 41) combined with “PIn” (reference numeral 42).

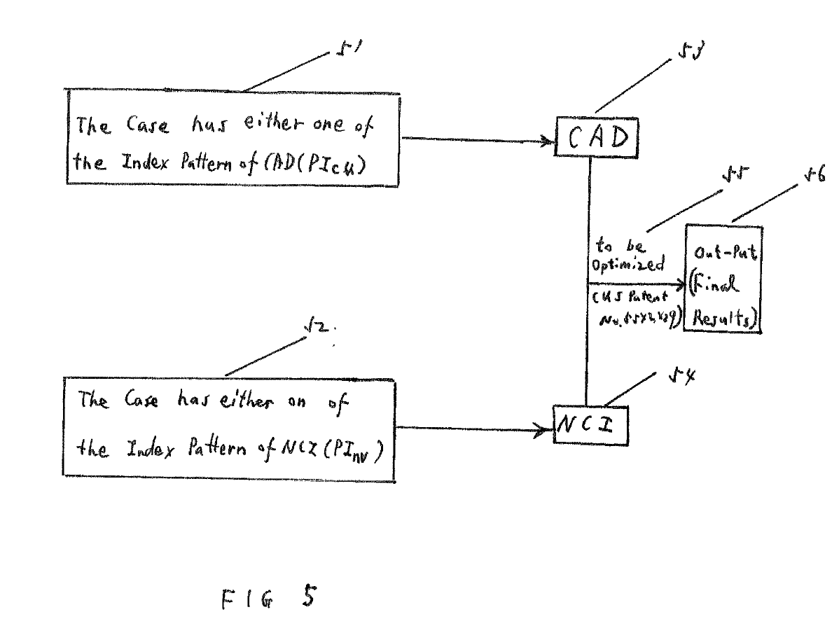

FIG. 5. Illustrated the process of differentiation of CAD vs NCI. Reference numeral 51 generally identifies the patient who's “positive index pattern” is within the scope of “PIc” group (i.e. pertains to the “PIc” group), he (the patient) shall be grossly diagnosed as CAD patient 53, that is the “gross diagnosis” of the patient. Reference numeral 52 generally identifies the patient who's “positive index pattern” is within the scope of “PIn” group (i.e. pertains to the “PIn” group), he (the patient) shall be grossly diagnosed as NCI patient 54, that is the “gross diagnosis” of the patient. Then use the “optimization process” (U.S. Pat. No. 5,542,429), to optimize the “gross diagnosis” 55, to finally get the out-put 56, that is the final results.

We claim:

1. A method of differentiation of Coronary Artery Disease (thereafter called “CAD”) vs Non-CAD-Ischemia (thereafter called CNCI”) comprises steps of
(a) acquiring sufficient amount of cases and getting every patient's positive (occurrence) “EMPI indexes”, according to the principle of the method and arrangements introduced in U.S. Pat. No. 5,509,425 and U.S. Pat. No. 5,649,544, as a “database”;
(b) using the “database”, empirically screening the “EMPI indexes” to acquire “CAD related indexes” as one “Group” (Cluster), i.e. “CAD related index group” called “Ic”, and acquiring another “index Group”, i.e. “NCI related index group”, called “In”, according to the difference of “positive (occurrence) rate of the indexes” in CAD patients and NCI patients, which one being higher;
(c) combining the said two “index groups' (“Ic” and “In”) to be one “Combined Index Group”, namely “Icn”, (i.e. Icn=Ic+In);
(d) using the “database”. empirically screening the patterns constructed by the said “Icn” of the EMPI indexes”, to two “Groups” according to the positive rate of the index patterns, of which the patterns with positive rate higher in CAD patients, called “Pc” Group, another Group with positive rate higher in NCI patients called “Pn” Group;
(e) using the “Pc” and “Pn” as a tool to grossly differentiate the patients to two categories: who having a positive index pattern(s) within the scope of “Pc” (i.e. pertains to “Pc”) to be identified as a “CAD patient”, vs. the patient who having a positive index pattern(s) within the scope of “Pn” to be identified as an “NCI patient”, that being “the gross differential diagnosis of the patient”; and
(f) using an “optimization process” (U.S. Pat. No. 5,542,429) to optimize the “gross diagnosis” mentioned in step (e) above, to get “final results”—a final differential diagnosis suggestion of CAD vs. NCI.

2. The method according to claim 1, wherein the step of “selecting the index group” is performed by a computerized “Data-Analysis program”.

3. The method according to claim 1, wherein the step of “selecting the index group” is performed by a computerized “Auto-Adjustment program”.

4. The method according to claim 1, wherein the step of “selecting the index group” is performed by a computerized “Neural-Network system”.

5. The method according to claim 1, wherein the step of “screening the index patterns” is performed by a computerized “Data-Analysis program”.

6. The method according to claim 1, wherein the step of “screening the index patterns” is performed by a computerized “Auto-Adjustment program”.

7. The method according to claim 1, wherein the step of “screening the index patterns” is performed by a computerized “Neural-Network system”.

8. An arrangement (means) of differentiation of Coronary Artery Disease (thereafter called “CAD”) vs Non-CAD-Ischemia (thereafter called “NCI”) comprises steps of
(a) means for acquiring sufficient amount of cases and getting every patient's positive (occurrence) “EMPI indexes”, according to the principle of the method and arrangements introduced in U.S. Pat. No. 5,509,425 and U.S. Pat. No. 5,649,544, as a “database”,
(b) means for using the “database”, empirically screening the “EMPI indexes” to acquire “CAD related indexes” as one “Group” (Cluster), i.e. “CAD related index group”, called “Ic”, and acquiring another “index Group”, i.e. “NCI related index group”, called “In”, according to the difference of “positive (occurrence) rate of the indexes” in CAD patients and NCI patients, which one being higher;
(c) means for combining the said two “index groups” (“Ic” and “In”) to be one “Combined Index Group”, namely “Icn”, (i.e. Icn=Ic+In);
(d) means for using the “database”, empirically screening the patterns constructed by the said “Icn” of the “EMPI indexes” to two Groups according to the positive rate of the index patterns, of which the patterns with positive rate higher in CAD patients, called “Pc” Group, another Group with the positive rate higher in NCI patients called “Pn” Group;
(e) means for using the “Pc” and “Pn” as a tool to grossly differentiate the patients to two categories: who having a positive index pattern(s) within the scope of “Pc” (i.e. pertains to “Pc”) to be identified as a “CAD patient”, vs. the patient who having a positive index pattern(s) within the scope of “Pn” to be identified as an “NCI patient”, that being “the gross differential diagnosis of the patient”; and
(f) means for using an “optimization process” (U.S. Pat. No. 5,542,429) to optimize the “gross diagnosis” mentioned in step (e) above, to get “final results”—a final differential diagnosis suggestion of CAD vs. NCI.
